(19) United States
(12) Patent Application Publication
(10) Pub. No.: US 2015/0254282 A1
(43) Pub. Date: Sep. 10, 2015

(54) MEDICAL IMAGE PROCESSING APPARATUS AND MEDICAL IMAGE PROCESSING METHOD

(71) Applicants: Kabushiki Kaisha Toshiba, Minato-ku (JP); Toshiba Medical Systems Corporation, Otawara-shi (JP)

(72) Inventor: Koji TAKEI, Nasushiobara (JP)

(73) Assignees: Kabushiki Kaisha Toshiba, Minato-ku (JP); Toshiba Medical Systems Corporation, Otawara-shi (JP)

(21) Appl. No.: 14/722,890

(22) Filed: May 27, 2015

Related U.S. Application Data
(63) Continuation of application No. PCT/JP2013/081945, filed on Nov. 27, 2013.

(30) Foreign Application Priority Data
Nov. 27, 2012 (JP) 2012-258975

(51) Int. Cl. G06F 17/30; G06T 15/40; G06T 11/00; G06T 15/20; A61B 6/03; A61B 6/00

(52) U.S. Cl. CPC G06F 17/30268; A61B 6/032; A61B 6/563; A61B 6/5205; A61B 6/503; G06T 11/003; G06T 15/205; G06T 15/40; G06T 2211/404

(57) ABSTRACT

A medical image processing apparatus according to embodiments includes identification information retrieving circuitry and medical image retrieving circuitry. The identification information retrieving circuitry retrieves identification information of a medical image serving as a basis of a three-dimensionally displayed image used for diagnosing a subject from object information that includes the identification information and setting information for three-dimensionally displaying the three-dimensionally displayed image. The medical image retrieving circuitry retrieves a medical image corresponding to retrieved identification information from storage circuitry that stores therein a plurality of medical images of a subject and identification information of the medical images in a corresponding manner.

### CROSS-REFERENCE TO RELATED APPLICATIONS

This application is a continuation of PCT international application Ser. No. PCT/JP2013/081945 filed on Nov. 27, 2013 which designates the United States, incorporated herein by reference, and which claims the benefit of priority from Japanese Patent Application No. 2012-258975, filed on Nov. 27, 2012, the entire contents of which are incorporated herein by reference.

### FIELD

Embodiments described herein relate generally to a medical image processing apparatus and a medical image processing method.

### BACKGROUND

In recent years, diagnoses using three-dimensional images are performed in medical sites such as hospitals. For example, medical image data of a plurality of axial surfaces along a body axial direction of a subject are captured by using medical image diagnosis devices such as an X-ray CT (Computed Tomography) device, an MRI (Magnetic Resonance Imaging), and an ultrasonic diagnosis device. Thereafter, mask processing of masking body parts other than a diagnosed part and a color information adjustment for artificially coloring the diagnosed part are performed to generate a three-dimensional image. A doctor observes the generated three-dimensional image to diagnose the subject.

To perform a diagnosis using a three-dimensional image, a large number of pieces of medical image data, for example, hundreds of pieces of medical image data are captured in one image-capturing operation. These pieces of medical image data are transmitted and received between devices via a network established in a medical site, or transmitted and received and while being stored in a DVD (Digital Versatile Disc).

### BRIEF DESCRIPTION OF THE DRAWINGS

FIG. 1 is an explanatory diagram of a configuration example of an image processing system according to a first embodiment;

FIG. 2 is a block diagram of a functional configuration of a medical image processing unit according to the first embodiment;

FIG. 3 is an example of information stored in an image storage unit;

FIG. 4 is an explanatory diagram of a data structure of object information;

FIG. 5 is an explanatory diagram of mask information;

FIG. 6 is an explanatory diagram of a process of generating a three-dimensional image by using object information;

FIG. 7 is an explanatory diagram of a process in which a reception unit receives a transmission request;

FIG. 8 is a flowchart for explaining a process procedure of the medical image processing unit according to the first embodiment;

FIGS. 9A and 9B are explanatory diagrams of effects of the medical image processing unit according to the first embodiment;

FIG. 10 is an explanatory diagram of a configuration example of an image processing system according to a second embodiment;

FIG. 11 is a block diagram of a functional configuration of a medical image processing unit according to the second embodiment;

FIG. 12 is a flowchart for explaining a process procedure of the medical image processing unit according to the second embodiment;

FIG. 13 is an explanatory diagram of effects of the medical image processing unit according to the second embodiment; and

FIG. 14 is another explanatory diagram of effects of the medical image processing unit according to the second embodiment.

### DETAILED DESCRIPTION

A medical image processing apparatus according to embodiments includes identification information retrieving circuitry and medical image retrieving circuitry. The identification information retrieving circuitry retrieves identification information of a medical image serving as a basis of a three-dimensionally displayed image used for diagnosing a subject from object information that includes the identification information and setting information for three-dimensionally displaying the three-dimensionally displayed image. The medical image retrieving circuitry retrieves a medical image corresponding to retrieved identification information from a storage circuitry that stores therein a plurality of medical images of a subject and identification information of the medical images in a corresponding manner.

Exemplary embodiments of a medical image processing apparatus and a medical image processing method will be explained below with reference to the accompanying drawings.

### First Embodiment

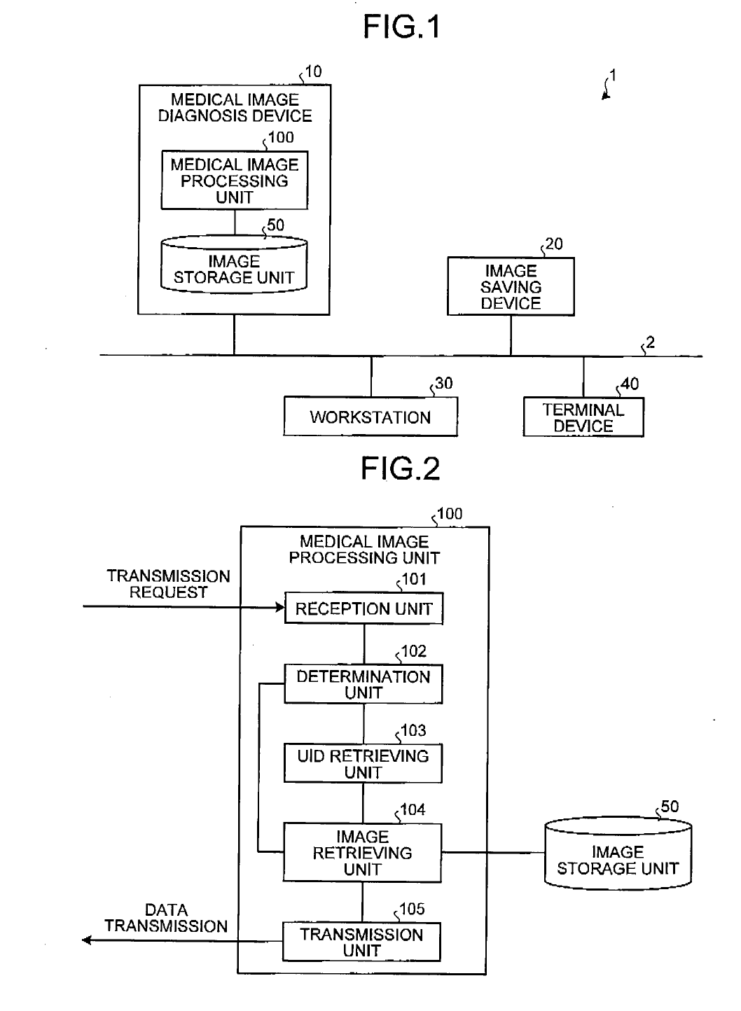

A configuration example of an image processing system according to a first embodiment is explained with reference to FIG. 1. FIG. 1 is an explanatory diagram of a configuration example of the image processing system according to the first embodiment. As shown in FIG. 1, a image processing system 1 includes a medical image diagnosis device 10, an image saving device 20, a workstation 30, and a terminal device 40. As shown in FIG. 1, these devices are directly or indirectly communicable with each other by, for example, an in-hospital LAN (Local Area Network) 2 that is installed in a hospital. For example, when a PACS (Picture Archiving and Communication System) is introduced in the image processing system 1, the respective devices transmit and receive medical image data and the like according to the DICOM (Digital Imaging and Communications in Medicine) standard.

The medical image diagnosis device 10 is an X-ray diagnosis device, an X-ray CT (Computed Tomography) device, an MRI (Magnetic Resonance Imaging) device, an ultrasonic diagnosis device, a SPECT (Single Photon Emission Computed Tomography) device, a PET (Positron Emission Computed Tomography) device, a SPECT-CT device in which a SPECT device is integrated with an X-ray CT device, a PET-CT device in which a PET device is integrated with an X-ray CT device, a group of these devices, and the like.

For example, the medical image diagnosis device 10 captures a subject to collect data such as projection data and MR signals, and generates a plurality of pieces of medical image data of axial surfaces along a body axial direction of the subject from the collected data. For example, the medical image diagnosis device 10 generates 500 pieces of medical image data of axial surfaces in one image-capturing operation. The medical image diagnosis device 10 stores a group of these 500 pieces of medical image data of axial surfaces in an image storage unit 50 (explained later) as volume data. Medical image data stored in the image storage unit 50 corresponds to a UID (Unique Identifier) serving as identification information of the medical image data, and both of them are stored in the image storage unit 50. Hereinafter, medical image data is also called “image data”.

The medical image diagnosis device 10 stores object information for three-dimensionally displaying image data in the image storage unit 50 along with a plurality of pieces of generated image data. This object information includes a UID of image data serving as a basis of a three-dimensional image (three-dimensionally displayed image) used for diagnosing a subject and setting information for three-dimensionally displaying a three-dimensional image. The medical image diagnosis device 10 also stores, for example, a patient ID for identifying a patient, an inspection ID for identifying an inspection, a device ID for identifying the medical image diagnosis device 10 that has performed an inspection, an image-capturing ID for identifying one image-capturing operation by the medical image diagnosis device 10, and the like in the image storage unit 50 as accompanying information. The medical image diagnosis device 10 can also store separately projection data and MR signals of a captured subject and the like themselves.

For example, the medical image diagnosis device 10 transmits a plurality of pieces of image data, object information, and accompanying information to the image saving device 20 and the workstation 30. As a result, the plural pieces of image data, the object information, and the accompanying information are stored in the image saving device 20. The plural pieces of image data are three-dimensionally displayed in the workstation 30.

The image saving device 20 is a database that saves image data. Specifically, the image saving device 20 stores image data generated by the medical image diagnosis device 10 and accompanying information thereof in a storage unit and saves them.

The workstation 30 is an image processing apparatus that performs image processing on image data. For example, the workstation 30 retrieves image data and accompanying information thereof from the medical image diagnosis device 10 or the image saving device 20, and three-dimensionally displays the retrieved image data on a monitor.

The terminal device 40 is a apparatus for doctors and medical technicians working in a hospital to observe image data. For example, the terminal device 40 is a PC (Personal Computer), a tablet PC, a PDA (Personal Digital Assistant), and a cell phone that are operated by doctors and medical technicians working in a hospital, and the like.

The medical image diagnosis device 10 includes the image storage unit 50 and a medical image processing unit 100. The image storage unit 50 stores therein a plurality of pieces of image data of a subject and UIDs thereof in a corresponding manner. Information stored in the image storage unit 50 is explained later.

The medical image processing unit 100 controls transmission and reception of volume data. For example, the medical image processing unit 100 receives a transmission request to transmit object information to the workstation 30. The medical image processing unit 100 retrieves a UID of image data serving as a basis of a three-dimensional image used for diagnosing a subject from object information corresponding to the received transmission request. The medical image processing unit 100 retrieves image data corresponding to the retrieved UID from the image storage unit 50. The medical image processing unit 100 transmits the retrieved image data and the object information to the workstation 30 corresponding to the transmission request. The medical image processing unit 100 can perform the same process as the process explained above, also when object information is transmitted to the image saving device 20.

A functional configuration of the medical image processing unit 100 according to the first embodiment is explained with reference to FIG. 2. FIG. 2 is a block diagram of a functional configuration of the medical image processing unit 100 according to the first embodiment. As shown in FIG. 2, the medical image processing unit 100 includes a reception unit 101, a determination unit 102, a UID retrieving unit 103, an image retrieving unit 104, and a transmission unit 105. The medical image processing unit 100 is connected to the image storage unit 50.

The image storage unit 50 stores therein a plurality of pieces of image data of a subject and UIDs thereof in a corresponding manner. For example, the image storage unit 50 stores therein 500 pieces of image data generated in one image-capturing operation and UIDs of the respective pieces of image data in a corresponding manner.

The image storage unit 50 stores therein object information for three-dimensionally displaying image data, in addition to a plurality of pieces of image data of a subject. The image storage unit 50 also stores therein, for example, a patient ID for identifying a patient, an inspection ID for identifying an inspection, a device ID for identifying the medical image diagnosis device 10 that has performed an inspection, an image-capturing ID for identifying one image-capturing operation by the medical image diagnosis device 10, and the like as accompanying information of the plural pieces of image data of a subject. Information stored in the image storage unit 50 is generated by doctors and medical technicians who use the medical image diagnosis device 10.

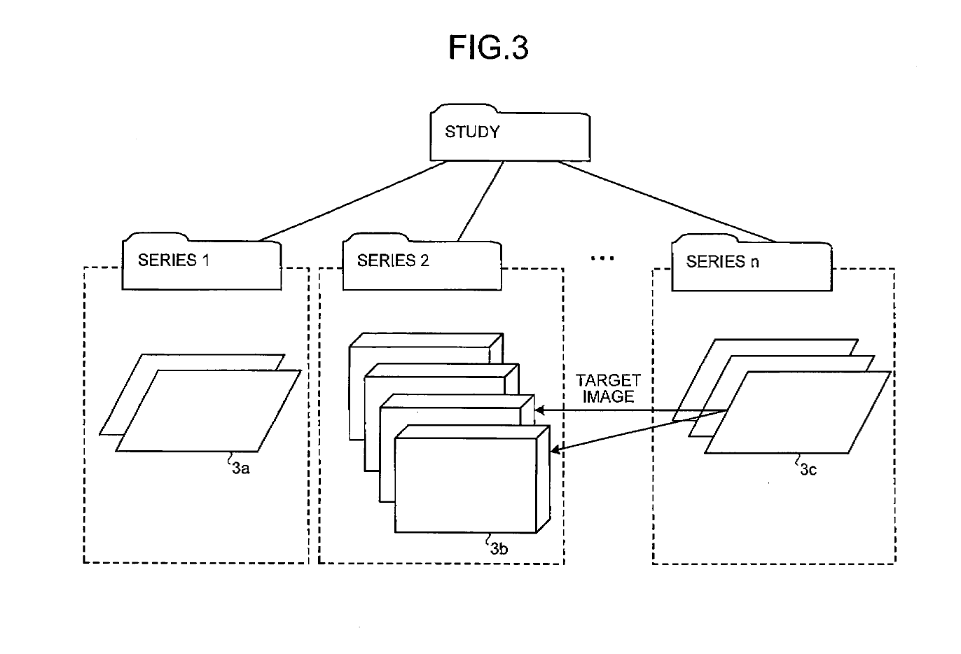

Information stored in the image storage unit 50 is explained with reference to FIG. 3. FIG. 3 is an example of information stored in the image storage unit 50. As shown in FIG. 3, the image storage unit 50 stores therein information retrieved by one inspection as “Study” that includes a plurality of pieces of “Series”. For example, the image storage unit 50 stores therein a scanogram 3a as “Series 1”. The scanogram 3a is information representing a captured part of a subject. The image storage unit 50 also stores therein volume data 3b “Series 2”. The volume data 3b is a group of image data of axial surfaces generated by one image-capturing operation. For example, when capturing is performed for a plurality of times in one inspection, the image storage unit 50 stores therein the volume data 3b corresponding to the number of image-capturing operations. Specifically, in a certain inspection, when the chest of a subject is captured and then the abdomen is captured, the volume data 3b of the chest and the volume data 3b of the abdomen are stored separately. Further, the image storage unit 50 stores therein object information 3c “Series n”. The object information 3c is information for three-dimensionally displaying a plurality of pieces of image data. For example, the object information 3c includes a UID of image data serving as a basis of a three-dimensional image used for diagnosing a subject and setting information for three-dimensionally displaying a three-dimensional image. In other words, a UID included in the object information 3c represents a target image of the volume data 3b serving as a basis of a three-dimensional image generated by using the object information 3c. For example, the object information 3c is generated for every diagnosis purpose. Specifically, when lungs are diagnosed, the object information 3c for three-dimensionally displaying image data of the lungs is generated. When the lungs and a breastbone are diagnosed, the object information 3c for three-dimensionally displaying image data of the lungs and the breastbone is generated. A UID can be identification information that represents a target image or can be identification information that represents all the target images (a plurality of target images). In the following explanations, a case where one UID represents one target image is explained; however, the present embodiment is not limited thereto.

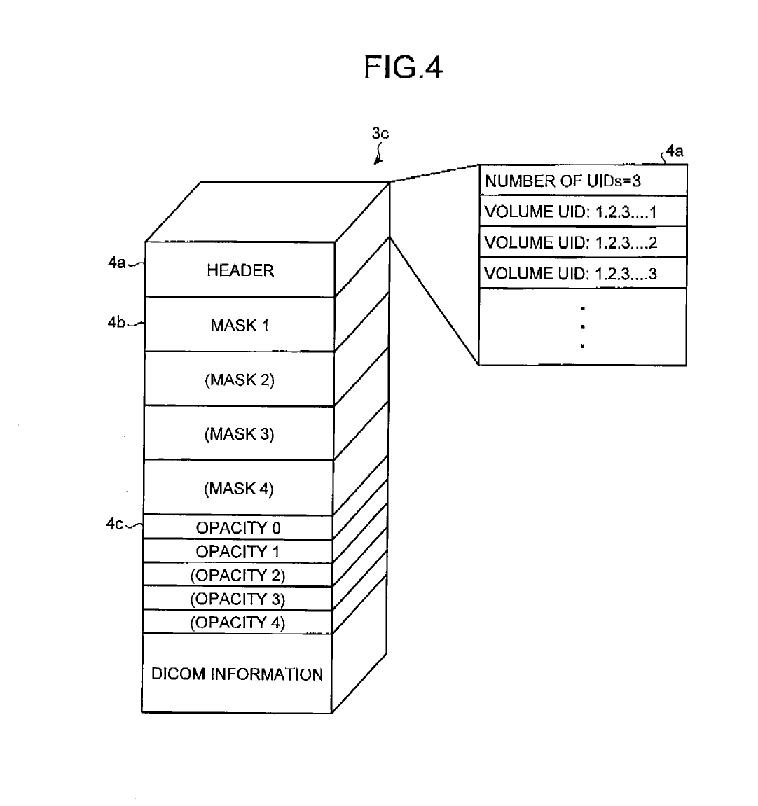

A data structure of the object information 3c is explained with reference to FIG. 4. FIG. 4 is an explanatory diagram of a data structure of the object information 3c. As shown in FIG. 4, the object information 3c includes Header information 4a, Mask information 4b, and Opacity information 4c. The Header information 4a is header information of the object information 3c and includes a UID of image data serving as a basis of a three-dimensional image generated by using the object information 3c. The Mask information 4b is mask information for three-dimensionally displaying image data. The Opacity information 4c is color information for coloring image data. DICOM information is information required for DICOM transmission (transmission based on the DICOM standard) of the object information 3c. For example, the DICOM information is DICOM additional tags.

As an example, there is a case where a three-dimensional image of a heart is generated by using, among 500 pieces of image data in which the chest of a subject is captured, 300 pieces of image data (target images). In this case, the object information 3c includes the respective UIDs of 300 pieces of image data used for generating the three-dimensional image of the heart in the Header information 4a. As another example, there is a case where a three-dimensional image of arteries is generated by using, among 500 pieces of image data in which the chest of a subject is captured, 50 pieces of image data. In this case, the object information 3c includes the respective UIDs of 50 pieces of image data used for generating the three-dimensional image of the arteries in the Header information 4a.

As still another example, there is a case where with respect to a piece of the object information 3c, image data included in a plurality of pieces of the volume data 3b, respectively are target images. For example, there is a case where a three-dimensional image of a torso is generated by using arbitrary image data from the volume data 3b of a chest and the volume data 3b of an abdomen. Specifically, a three-dimensional image of the torso is generated by using 300 pieces of image data among 500 pieces of image data in which the chest is captured and 350 pieces of image data among 600 pieces of image data in which the abdomen is captured. In this case, the object information 3c includes the respective UIDs of 300 pieces of image data for generating a three-dimensional image of the chest and the respective UIDs of 350 pieces of image data for generating a three-dimensional image of the abdomen in the Header information 4a.

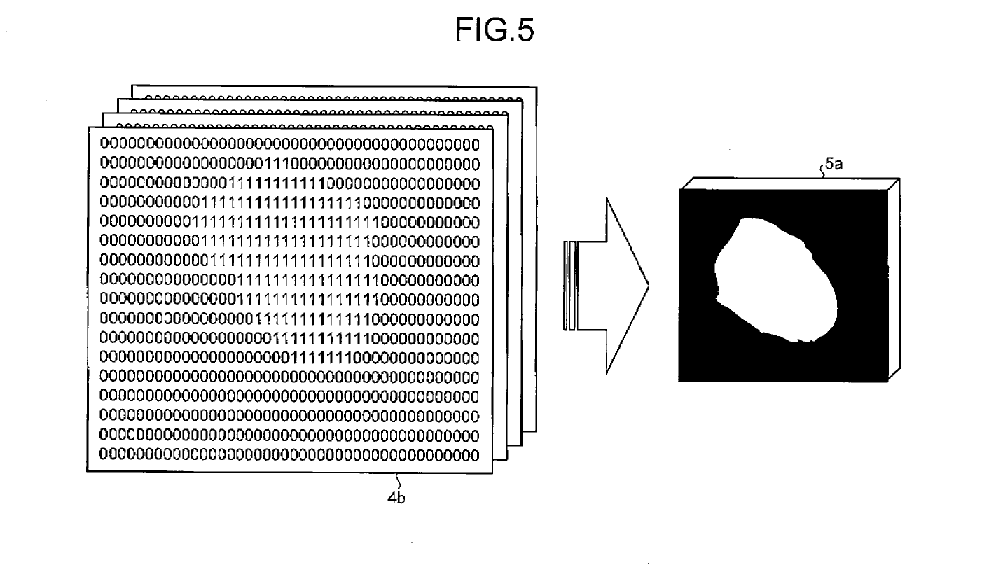

Mask information is explained with reference to FIG. 5. FIG. 5 is an explanatory diagram of mask information. As shown in FIG. 5, the Mask information 4b is mask information in which, with respect to image data to be three-dimensionally displayed, a pixel that is three-dimensionally displayed is “1” and a pixel that is not three-dimensionally displayed is “0”. That is, the Mask information 4b is used for generating a three-dimensional image 5a from volume data by multiplying the corresponding pixels of the respective pieces of image data to be three-dimensionally displayed by “1” or “0”.

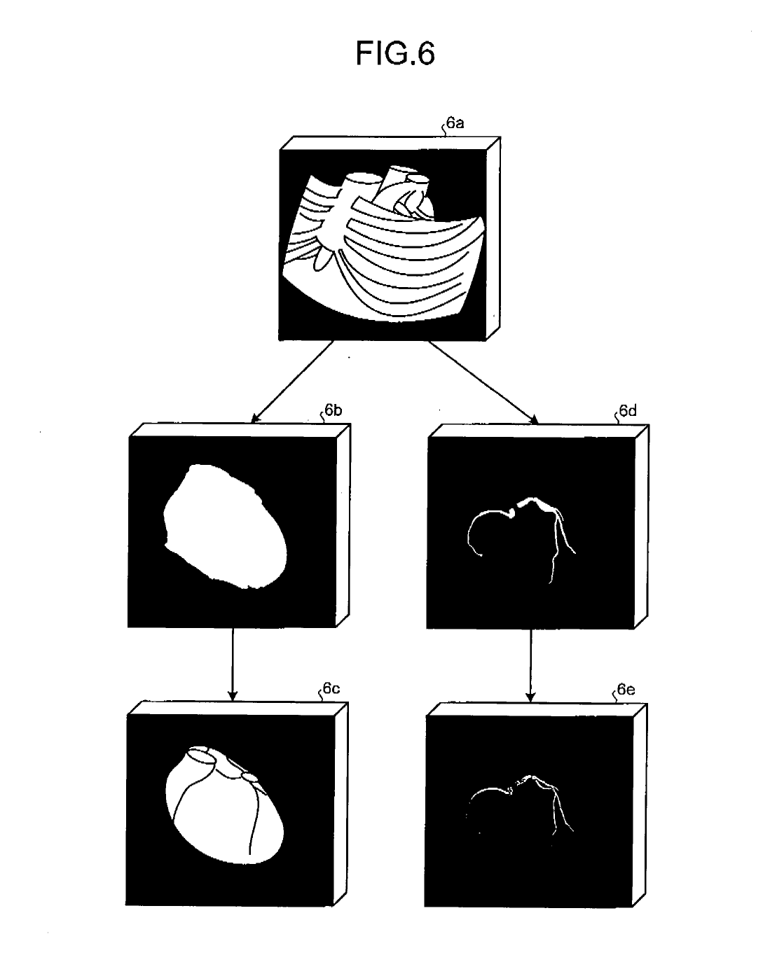

A process of generating a three-dimensional image by using object information is explained with reference to FIG. 6. FIG. 6 is an explanatory diagram of a process of generating a three-dimensional image by using object information. As shown in FIG. 6, volume data 6a in which the chest of a subject is captured becomes a three-dimensional image 6b by using the Mask information 4b for three-dimensionally displaying only a heart. The three-dimensional image 6b is then colored by using the Opacity information 4c for three-dimensionally displaying the heart to become a three-dimensional image 6c. Meanwhile, the volume data 6a also becomes a three-dimensional image 6d by using the Mask information 4b for three-dimensionally displaying only arteries. The three-dimensional image 6d is then colored by using the Opacity information 4c for three-dimensionally displaying the arteries to become a three-dimensional image 6e.

Returning to the explanation of FIG. 2, the reception unit 101 receives a transmission request to transmit object information to the workstation 30. The reception unit 101 transmits object information corresponding to the received transmission request to the determination unit 102.

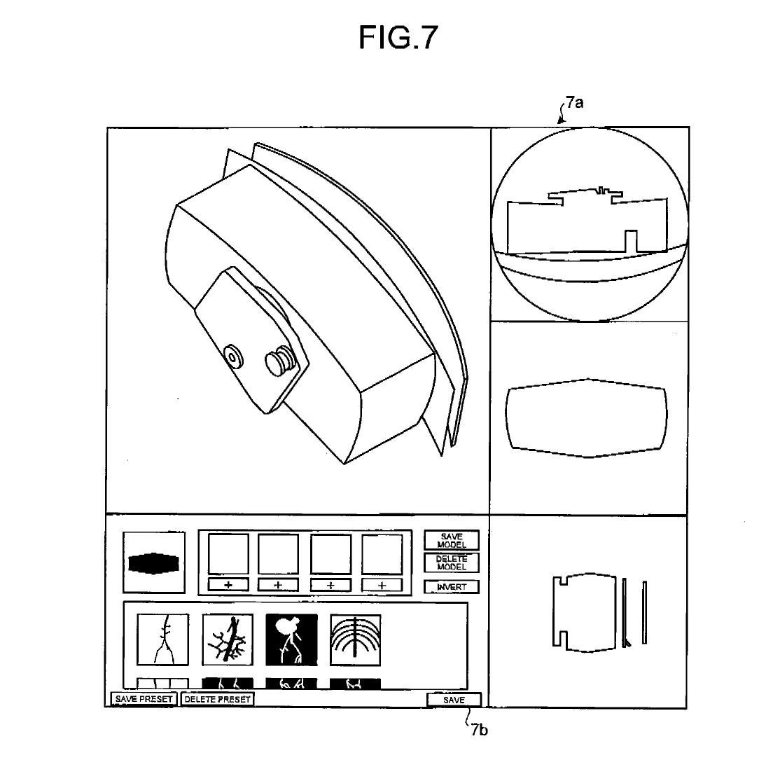

A process in which the reception unit 101 receives a transmission request is explained with reference to FIG. 7. FIG. 7 is an explanatory diagram of a process in which the reception unit 101 receives a transmission request. FIG. 7 shows a display screen 7a that is displayed on a monitor of the medical image diagnosis device 10. The display screen 7a is a screen that is used by doctors and medical technicians who use the medical image diagnosis device 10 to generate a three-dimensional image used for diagnosing a subject. A doctor or medical technician adjusts object information for generating a three-dimensional image from volume data of the subject by using the display screen 7a. FIG. 7 exemplifies a case where object information is adjusted by using a phantom. Specifically, among the volume data, an UID of image data serving as a basis of a three-dimensional image used for diagnosing the subject is specified by the doctor or medical technician, and the UID of the specified image data is registered in the Header information 4a of the object information. Further, mask information for generating a three-dimensional image from the specified image data is adjusted by the doctor or medical technician, and the adjusted mask information is registered as the Mask information 4b. Color information for coloring a three-dimensional image generated by the mask information is adjusted by the doctor or medical technician, and the adjusted color information is registered as the Opacity information 4c. When adjustments of the object information are completed, the doctor or medical technician presses a Save button 7b to store the object information in the image storage unit 50. When the Save button 7b is pressed by the doctor or medical technician, the reception unit 101 receives a transmission request to transmit the adjusted object information to the workstation 30. The reception unit 101 transmits object information corresponding to the received transmission request to the determination unit 102.

Alternatively, the reception unit 101 can receive a transmission request to transmit image data specified by the doctor or medical technician along with object information. In this case, the reception unit 101 receives a transmission request that includes a UID of the image data specified by the doctor or medical technician and the object information. The reception unit 101 transmits the object information and the UID of the image data corresponding to the received transmission request to the determination unit 102.

The determination unit 102 determines whether a transmission request received by the reception unit 101 is a transmission request for object information. For example, when the determination unit 102 receives only object information from the reception unit 101, the determination unit 102 transmits the received object information to the UID retrieving unit 103. On the other hand, when the determination unit 102 receives object information and a UID of image data from the reception unit 101, the determination unit 102 transmits the received object information and the received UID of the image data to the image retrieving unit 104.

The UID retrieving unit 103 retrieves a UID of image data (a target image) serving as a basis of a three-dimensional image used for diagnosing a subject from object information corresponding to a transmission request. For example, the UID retrieving unit 103 receives object information from the determination unit 102. The UID retrieving unit 103 retrieves a UID of image data from header information of the received object information. The UID retrieving unit 103 transmits the retrieved UID and the object information to the image retrieving unit 104.

As an example, there is a case where a three-dimensional image of a heart is generated by using, among 500 pieces of image data in which the chest of a subject is captured, 300 pieces of image data. In this case, the UID retrieving unit 103 retrieves the respective UIDs of 300 pieces of image data serving as a basis of the three-dimensional image of the heart from header information of object information. As another example, there is a case where a three-dimensional image of arteries is generated by using, among 500 pieces of image data in which the chest of a subject is captured, 50 pieces of image data. In this case, the UID retrieving unit 103 retrieves the respective UIDs of 50 pieces of image data serving as a basis of the three-dimensional image of the arteries.

The image retrieving unit 104 retrieves image data (a target image) corresponding to a UID from the image storage unit 50. For example, the image retrieving unit 104 receives a UID and object information from the determination unit 102 or the UID retrieving unit 103. The image retrieving unit 104 retrieves image data corresponding to the received UID from the image storage unit 50. The image retrieving unit 104 transmits the retrieved image data and the object information to the transmission unit 105.

As an example, the image retrieving unit 104 receives the respective UIDs of 300 pieces of image data serving as a basis of a three-dimensional image of the heart from the UID retrieving unit 103. The image retrieving unit 104 retrieves 300 pieces of image data corresponding to the received respective UIDs from the image storage unit 50. The image retrieving unit 104 transmits 300 pieces of image data that have been retrieved and object information to the transmission unit 105.

The transmission unit 105 transmits retrieved image data (an retrieved target image) and object information to the workstation 30 corresponding to a transmission request. For example, the transmission unit 105 receives image data and object information from the image retrieving unit 104. The transmission unit 105 transmits the received image data and object information to the workstation 30.

As an example, the transmission unit 105 receives 300 pieces of image data serving as a basis of a three-dimensional image of the heart and object information from the image retrieving unit 104. The transmission unit 105 transmits 300 pieces of image data that have been received and the received object information to the workstation 30.

For example, the transmission unit 105 transmits object information and retrieved image data according to the DICOM standard. For example, the transmission unit 105 converts object information into a data format called “Segmentation” of the DICOM standard based on information of DICOM additional tags of the object information. The transmission unit 105 also converts retrieved image data into a data format called “Enhanced CT Image Storage” of the DICOM standard. The transmission unit 105 then transmits the object information and the image data that have been converted into the respective data formats of the DICOM standard to the workstation 30 corresponding to a transmission request.

For example, functions of the medical image processing unit 100 can be realized by executing a predetermined program by an integration circuit such as an ASIC (Application Specific Integrated Circuit) and an FPGA (Field Programmable Gate Array), or a CPU (Central Processing Unit).

For example, the image storage unit 50 corresponds to a semiconductor memory element such as a RAM (Random Access Memory) and a flash memory or a storage device such as a hard disk device and an optical disk device.

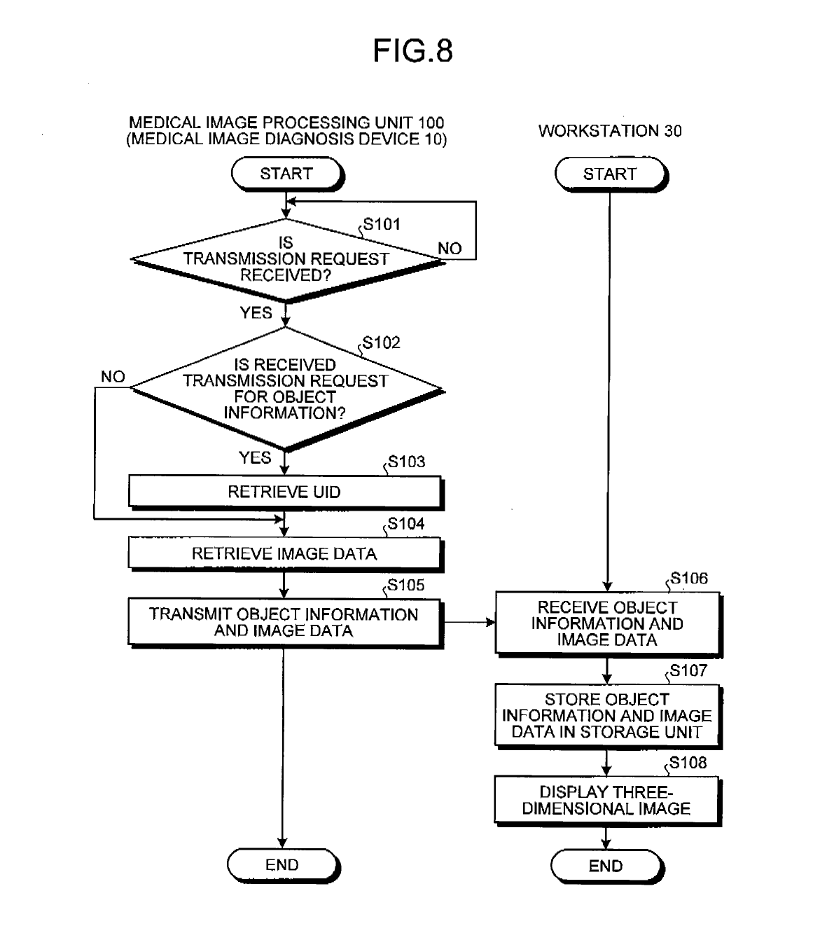

Next, a process procedure of the medical image processing unit 100 according to the first embodiment is explained with reference to FIG. 8. FIG. 8 is a flowchart for explaining a process procedure of the medical image processing unit 100 according to the first embodiment. A process shown in FIG. 8 is performed by, for example, the reception unit 101 receiving a transmission request.

As shown in FIG. 8, when the reception unit 101 receives a transmission request (YES at Step S101), the determination unit 102 determines whether this transmission request is a transmission request for object information (Step S102). Until the reception unit 101 receives a transmission request (NO at Step S101), the medical image processing unit 100 is in a standby state.

When the transmission request is a transmission request for object information (YES at Step S102), the determination unit 102 transmits object information to the UID retrieving unit 103. The UID retrieving unit 103 retrieves a UID of image data (a target image) serving as a basis of a three-dimensional image used for diagnosing a subject from the object information corresponding to the transmission request (Step S103). When the transmission request is a transmission request for object information and a UID of image data (NO at Step S102), the medical image processing unit 100 shifts the process to Step S104.

The image retrieving unit 104 then retrieves image data corresponding to the respective UIDs from the image storage unit 50 (Step S104). The transmission unit 105 transmits the object information and the image data to the workstation 30 corresponding to the transmission request (Step S105).

The workstation 30 receives object information and image data (Step S106). The workstation 30 stores the received object information and image data in a storage unit of the workstation 30 itself (Step S107). The workstation 30 displays a three-dimensional image based on the object information and the image data (Step S108).

Next, effects of the medical image processing unit 100 according to the first embodiment are explained. The medical image processing unit 100 receives a transmission request to transmit object information to the workstation 30. The medical image processing unit 100 retrieves a UID of image data (a target image) serving as a basis of a three-dimensional image used for diagnosing a subject from object information corresponding to the received transmission request. The medical image processing unit 100 retrieves image data corresponding to the retrieved UID from the image storage unit 50. The medical image processing unit 100 transmits the retrieved image data and the object information to the workstation 30 corresponding to the transmission request. Accordingly, the medical image processing unit 100 can realize a diagnosis in the workstation 30 immediately after an inspection is finished.

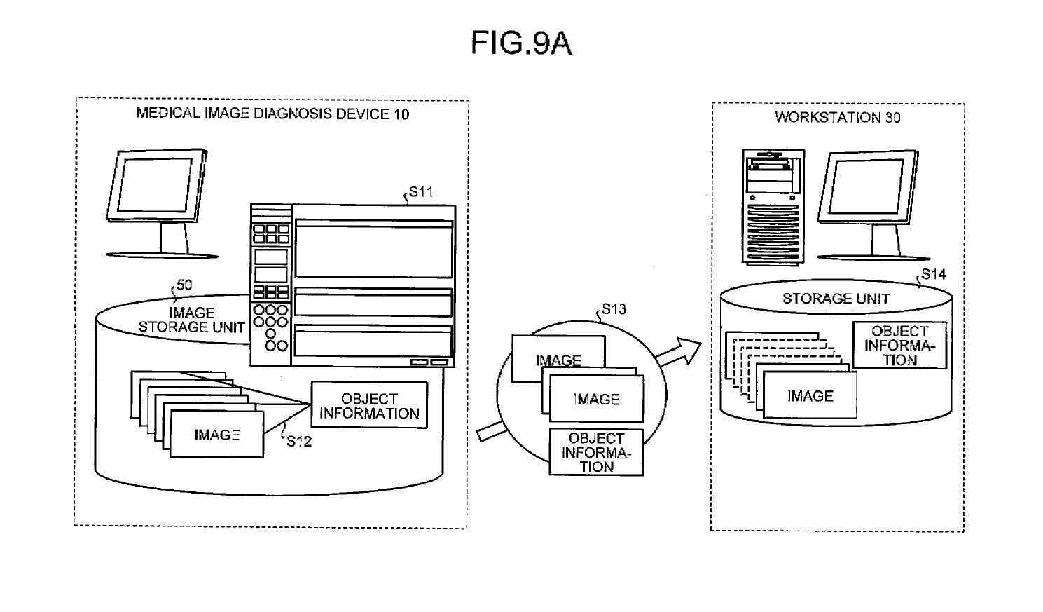

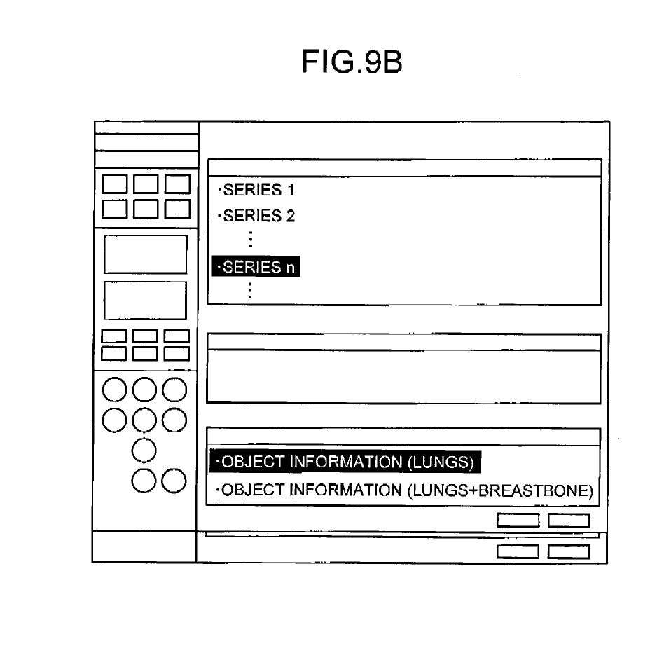

Effects of the medical image processing unit 100 according to the first embodiment are explained with reference to FIG. 9A. FIG. 9A is an explanatory diagram of effects of the medical image processing unit 100 according to the first embodiment. As shown in FIG. 9A, when the Save button 7b is pressed by a doctor or medical technician, the medical image processing unit 100 receives a transmission request to transmit object information that has been adjusted to the workstation 30 (S11). The medical image processing unit 100 retrieves a UID of image data serving as a basis of a three-dimensional image used for diagnosing a subject from object information corresponding to the transmission request, and retrieves image data corresponding to each of the retrieved UIDs from the image storage unit 50 (S12). The medical image processing unit 100 transmits the retrieved image data and the object information to the workstation 30 (S13). When the workstation 30 receives the object information and the image data, the workstation 30 stores the received object information and image data in a storage unit of the workstation 30 itself (S14). The workstation 30 then displays a three-dimensional image based on the object information and the image data. As explained above, by simply receiving a transmission request for object information, the medical image processing unit 100 can transmit image data serving as a basis of a three-dimensional image used for diagnosing a subject and object information to the workstation 30. As a result, because the medical image processing unit 100 can transfer image data used for a diagnosis at a higher speed as compared to a case where all pieces of image data are transmitted, it is possible to perform a diagnosis immediately after an inspection is finished.

### Modification of the First Embodiment

While the first embodiment has explained a case where, at the time of saving object information that has been adjusted, a target image (image data) of the object information is transmitted to the workstation 30, the embodiments are not limited thereto. For example, when there are a plurality of pieces of object information that have been adjusted, an operator can specify arbitrary object information, receive an instruction to transmit the object information to the workstation 30, and transmit a target image of the specified object information.

FIG. 9B is an explanatory diagram of a modification of the medical image processing unit according to the first embodiment. FIG. 9B is an example of a display screen exemplified at S11 of FIG. 9A. For example, in FIG. 9B, the medical image diagnosis device 10 displays a list of a plurality of “Series” for an inspection (Study) that has already been performed. When an operator (a doctor or medical technician) selects “Series n” from this list, the medical image processing unit 100 displays a list of object information stored in “Series n”. According to the example of FIG. 9B, the medical image processing unit 100 displays a list that includes “object information (lungs)” and “object information (lungs+breastbone)”. These pieces of object information are generated from volume data of the chest and the abdomen of a subject captured in an inspection according to different diagnosis purposes. Specifically, “object information (lungs)” is object information for three-dimensionally displaying image data of lungs, and “object information (lungs+breastbone)” is object information for three-dimensionally displaying image data of the lungs and a breastbone.

When arbitrary object information (for example, “object information (lungs)”) is selected from the list and a transmission button is pressed by the operator, the medical image processing unit 100 receives a transmission request to transmit the selected object information to the workstation 30 (S11). Thereafter, the medical image processing unit 100 transmits a target image of the object information by the procedure that has been explained with reference to FIG. 9A.

### Second Embodiment

Next, a second embodiment is explained. The first embodiment has explained a case where, at the time of transmitting object information from the medical image diagnosis device 10 to the workstation 30, image data serving as a basis of a three-dimensional image that is generated by the object information is also transmitted. However, the present embodiment is not limited thereto. For example, when object information is received by the workstation 30, image data serving as a basis of a three-dimensional image that is generated by the object information can be retrieved. For example, there is a case where the workstation 30 receives object information from a DVD or the medical image diagnosis device 10. The second embodiment explains a process in a case where, when object information is received by the workstation 30, image data serving as a basis of a three-dimensional image that is generated by the object information is retrieved.

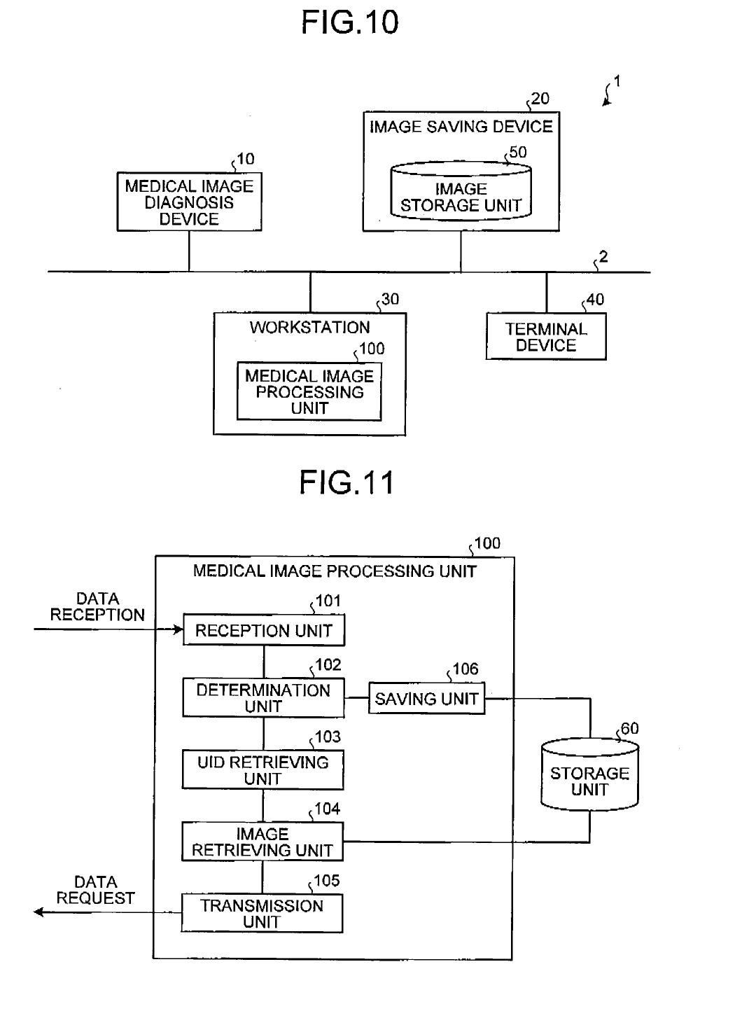

A configuration example of an image processing system according to the second embodiment is explained with reference to FIG. 10. FIG. 10 is an explanatory diagram of a configuration example of the image processing system according to the second embodiment. As shown in FIG. 10, the image processing system 1 includes the medical image diagnosis device 10, the image saving device 20, the workstation 30, and the terminal device 40, similarly to FIG. 1.

As shown in FIG. 10, the image saving device 20 according to the second embodiment is different from the image saving device 20 shown in FIG. 1 in that the image saving device 20 according to the second embodiment includes the image storage unit 50. The workstation 30 according to the second embodiment is different from the workstation 30 shown in FIG. 1 in that the workstation 30 according to the second embodiment includes the medical image processing unit 100. Like reference signs are denoted to functional units that exhibit functions identical to those of the embodiments explained above, and explanations thereof will be omitted.

The medical image processing unit 100 receives object information from outside thereof. The medical image processing unit 100 retrieves a UID of image data serving as a basis of a three-dimensional image used for diagnosing a subject from the received object information. The medical image processing unit 100 retrieves image data corresponding to the retrieved UID from the image storage unit 50.

A functional configuration of the medical image processing unit 100 according to the second embodiment is explained with reference to FIG. 11. FIG. 11 is a block diagram of a functional configuration of the medical image processing unit 100 according to the second embodiment. As shown in FIG. 11, the medical image processing unit 100 includes the reception unit 101, the determination unit 102, the UID retrieving unit 103, the image retrieving unit 104, the transmission unit 105, and a saving unit 106. The medical image processing unit 100 is connected to a storage unit 60.

The storage unit 60 is a storage device that is provided within the workstation 30 in order that the workstation 30 performs image processing on image data. For example, the storage unit 60 corresponds to a semiconductor memory element such as a RAM (Random Access Memory) and a flash memory or a storage device such as a hard disk device and an optical disk device.

The reception unit 101 receives object information from outside of the medical image processing unit 100. For example, the reception unit 101 receives object information via a DVD drive device connected to the workstation 30 by Q/R Move (Query/Retrieve Move) from a DVD having the object information stored therein. The Q/R Move is a protocol for retrieving specified data from a connected device. The reception unit 101 transmits the received object information to the determination unit 102. At this time, the reception unit 101 can receive, along with object information, image data serving as a basis of a three-dimensional image that is generated by the object information.

Furthermore, for example, the reception unit 101 receives object information by the Q/R move from the medical image diagnosis device 10 connected to the workstation 30. The reception unit 101 transmits the received object information to the determination unit 102. At this time, the reception unit 101 can receive, along with object information, image data serving as a basis of a three-dimensional image that is generated by the object information.

Further, for example, the reception unit 101 receives image data transmitted from the image saving device 20. The reception unit 101 transmits the received image data to the determination unit 102.

The determination unit 102 determines whether information received by the reception unit 101 includes image data. For example, when the determination unit 102 receives only object information from the reception unit 101, the determination unit 102 transmits the received object information to the UID retrieving unit 103. When the determination unit 102 receives image data from the reception unit 101, the determination unit 102 transmits the received image data to the saving unit 106, Further, when the determination unit 102 receives object information and image data from the reception unit 101, the determination unit 102 transmits the received object information and image data to the saving unit 106.

The image retrieving unit 104 retrieves image data corresponding to a UID from the image storage unit 50. For example, the image retrieving unit 104 receives a UID and object information from the UID retrieving unit 103. The image retrieving unit 104 determines whether image data corresponding to the received UID is present in the storage unit 60 of the medical image processing unit 100 itself. When image data corresponding to the received UID is present in the storage unit 60 of the medical image processing unit 100 itself, the image retrieving unit 104 retrieves the image data from the storage unit 60. On the other hand, when image data corresponding to the received UID is not present in the storage unit 60 of the medical image processing unit 100 itself, the image retrieving unit 104 generates a request to retrieve the image data. The image retrieving unit 104 transmits the generated retrieving request via the transmission unit 105 to the image saving device 20.

As an example, the image retrieving unit 104 receives the respective UIDs of 300 pieces of image data serving as a basis of a three-dimensional image of a heart from the UID retrieving unit 103. When 300 pieces of image data corresponding to the respective received UIDs are not present in the storage unit 60, the image retrieving unit 104 generates a request to retrieve 300 pieces of image data. The image retrieving unit 104 then transmits the generated retrieving request via the transmission unit 105 to the image saving device 20.

The transmission unit 105 transmits an retrieving request to retrieve image data to the image saving device 20. For example, the transmission unit 105 receives an retrieving request to retrieve image data from the image retrieving unit 104. The transmission unit 105 transmits the received retrieving request to the image saving device 20.

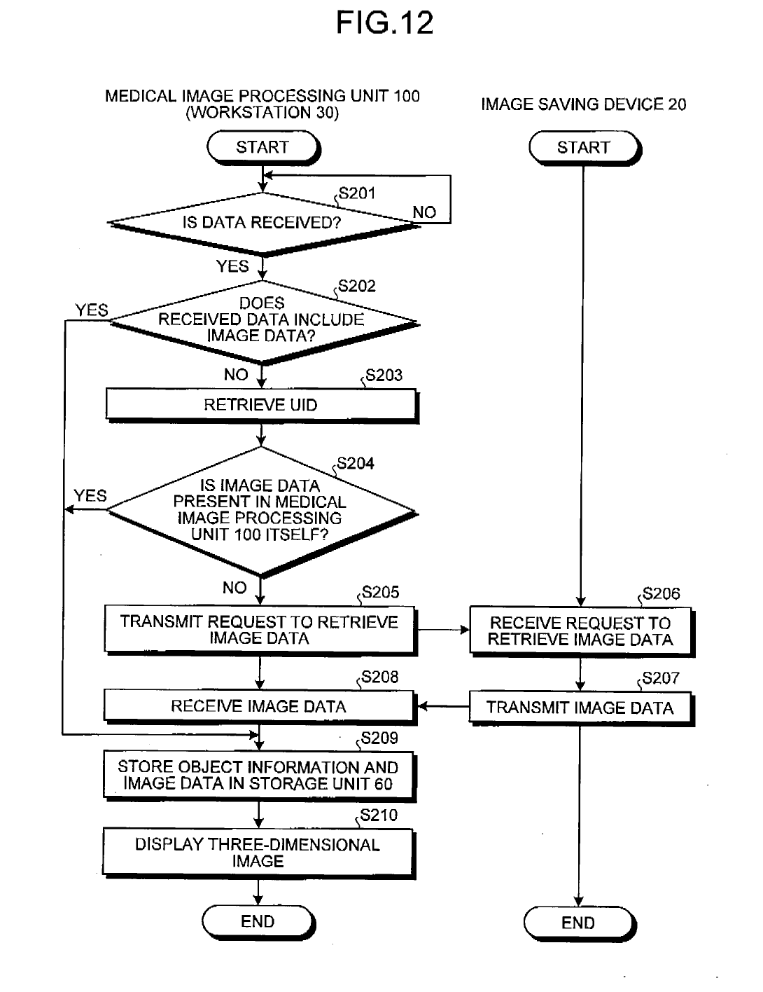

Next, a process procedure of the medical image processing unit 100 according to the second embodiment is explained with reference to FIG. 12. FIG. 12 is a flowchart for explaining a process procedure of the medical image processing unit 100 according to the second embodiment. A process shown in FIG. 12 is performed by, for example, the reception unit 101 receiving data.

As shown in FIG. 12, when the reception unit 101 receives data (YES at Step S201), the determination unit 102 determines whether the received data includes image data (Step S202). Until the reception unit 101 receives data (NO at Step S201), the medical image processing unit 100 is in a standby state.

When image data is not included (NO at Step S202), the determination unit 102 transmits object information to the UID retrieving unit 103. The UID retrieving unit 103 then retrieves a UID of image data serving as a basis of a three-dimensional image used for diagnosing a subject from the object information (Step S203).

The image retrieving unit 104 then determines whether image data corresponding to a UID is present in the storage unit 60 of the medical image processing unit 100 itself (Step S204). When the image data is not present in the medical image processing unit 100 itself (NO at Step S204), the image retrieving unit 104 transmits a request to retrieve image data transmits via the transmission unit 105 to the image saving device 20 (Step S205).

When the image saving device 20 receives a request to retrieve image data (Step S206), the image saving device 20 retrieves image data corresponding to the retrieving request from the image storage unit 50. The image saving device 20 then transmits the retrieved image data to the medical image processing unit 100 (Step S207).

When the reception unit 101 receives image data from the image saving device 20 (Step S208), the reception unit 101 stores the received image data and the corresponding object information in the storage unit 60 (Step S209). The medical image processing unit 100 then generates a three-dimensional image based on the retrieved image data and object information, and displays the three-dimensional image (Step S210).

When image data is included (YES at Step S202), the medical image processing unit 100 shifts the process to Step S209. When image data is not included in the medical image processing unit 100 itself (NO at Step S204), the image retrieving unit 104 transmits a request to retrieve image data via the transmission unit 105 to the image saving device 20 (Step S205).

Next, effects of the medical image processing unit 100 according to the second embodiment are explained. The medical image processing unit 100 receives object information from outside thereof. The medical image processing unit 100 retrieves a UID of image data serving as a basis of a three-dimensional image used for diagnosing a subject from the received object information. The medical image processing unit 100 retrieves image data corresponding to the retrieved UID from the image storage unit 50. Accordingly, the medical image processing unit 100 can realize a diagnosis in the medical image processing unit 100 itself immediately after an inspection is finished.

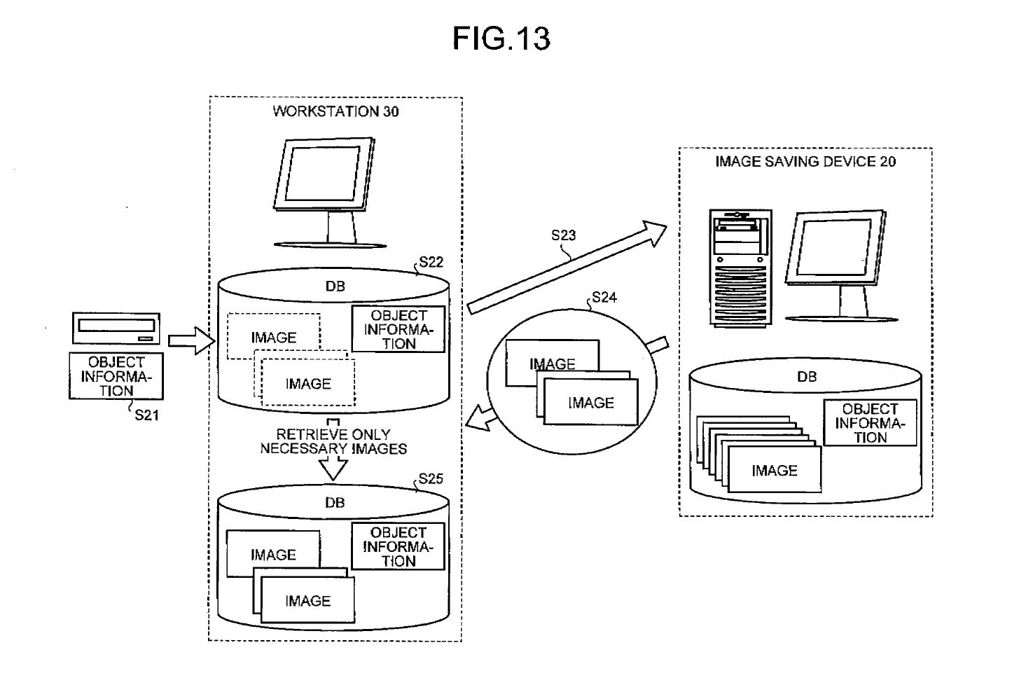

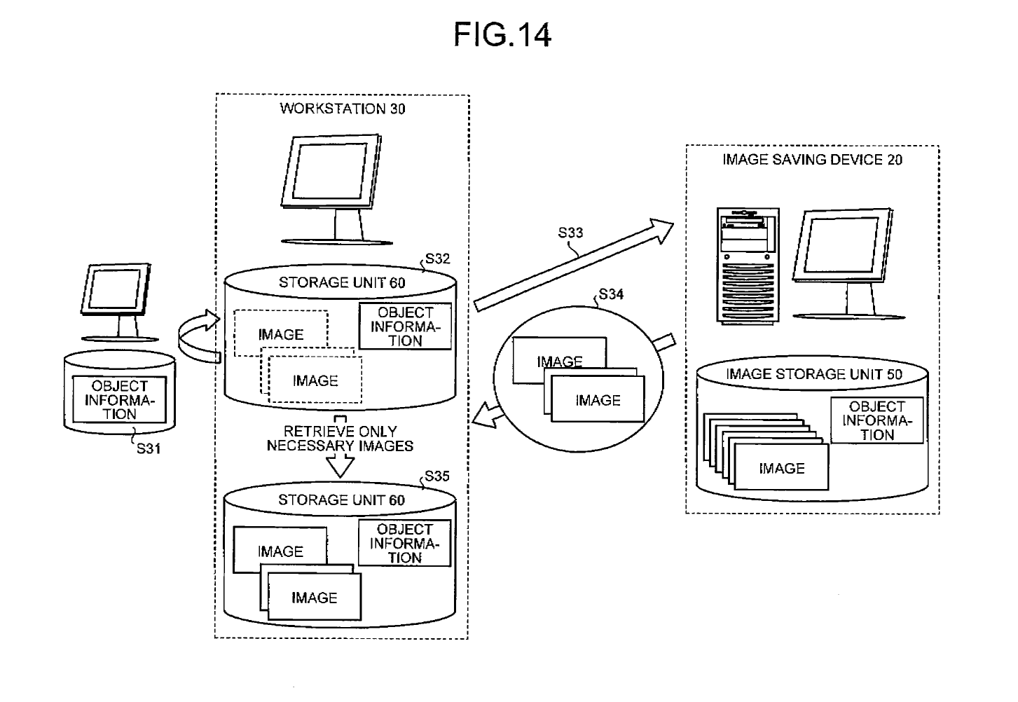

Effects of the medical image processing unit 100 according to the second embodiment are explained with reference to FIGS. 13 and 14. FIGS. 13 and 14 are explanatory diagrams of effects of the medical image processing unit 100 according to the second embodiment. As shown in FIG. 13, the medical image processing unit 100 receives object information via a DVD drive device connected to the workstation 30 by Q/R Move from a DVD having the object information stored therein (S21). The medical image processing unit 100 retrieves a UID from the received object information, and determines whether image data corresponding to the retrieved UID is present in the storage unit 60 of the medical image processing unit 100 itself (S22). When the image data is not present in the medical image processing unit 100 itself, the medical image processing unit 100 transmits a request to retrieve image data via the transmission unit 105 to the image saving device 20 (523). The medical image processing unit 100 receives image data corresponding to this retrieving request from the image saving device 20 (S24), thereby retrieving only image data serving as a basis of a three-dimensional image that is generated by the object information (825). As a result, because the medical image processing unit 100 can receive image data used for a diagnosis at a higher speed as compared to a case where all pieces of image data are received, it is possible to perform a diagnosis in the medical image processing unit 100 itself immediately after an inspection is finished.

Also in a case where object information is stored in a DVD, the object information is recorded (stored) according to the DICOM standard, similarly to a case of communication. For example, the object information is converted into a DICOM media format, which is the DICOM standard for storage media. In this case, object information that has been converted into a data format called “Segmentation” of the DICOM standard and a DICOMDIR file are stored in a DVD. The DICOMDIR file corresponds to index information of information stored in a DVD.

As shown in FIG. 14, the medical image processing unit 100 receives object information by Q/R Move from the medical image diagnosis device 10 connected to the workstation 30 (S31). The medical image processing unit 100 retrieves a UID from the received object information, and determines whether image data corresponding to the retrieved UID is present in the storage unit 60 of the medical image processing unit 100 itself (S32). When the image data is not present in the medical image processing unit 100 itself, the medical image processing unit 100 transmits a request to retrieve image data via the transmission unit 105 to the image saving device 20 (S33). The medical image processing unit 100 receives image data corresponding to this retrieving request from the image saving device 20 (S34), thereby retrieving only image data serving as a basis of a three-dimensional image that is generated by the object information (S35). As a result, because the medical image processing unit 100 can receive image data used for a diagnosis at a higher speed as compared to a case where all pieces of image data are received, it is possible to perform a diagnosis in the medical image processing unit 100 itself immediately after an inspection is finished.

In the embodiments described above, a case where object information is recorded in a DVD has been explained as an example of a portable recording medium; however, the embodiments are not limited thereto. That is, the storage medium is not limited to a DVD, and for example, object information can be stored in other types of portable recording media such as a USB (Universal Serial Bus) memory or an MD (MiniDisc).

While the above embodiments have explained a case where one UID represents one target image, the embodiments are not limited thereto. For example, one UID can represent all the target images (a plurality of target images). Specifically, in the medical image processing unit 100, the reception unit 101 receives a transmission request to transmit object information to the workstation 30. The UID retrieving unit 103 retrieves a UID of image data (target images) serving as a basis of a three-dimensional image used for diagnosing a subject from object information corresponding to the received transmission request. The UID represents all the target images. The image retrieving unit 104 retrieves all the target images corresponding to this UID from the image storage unit 50. The medical image processing unit 100 transmits the retrieved target images and the object information to the workstation 30 corresponding to the transmission request. As a result, the medical image processing unit 100 can realize a diagnosis in the workstation 30 immediately after an inspection is finished.

At least one of the embodiments described above, it is possible to perform a diagnosis immediately after an inspection is finished.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising:
identification information retrieving circuitry that retrieves identification information of a medical image serving as a basis of a three-dimensionally displayed image used for diagnosing a subject from object information that includes the identification information and setting information for three-dimensionally displaying the three-dimensionally displayed image; and
medical image retrieving circuitry that retrieves a medical image corresponding to identification information retrieved by the identification information retrieving circuitry from storage circuitry that stores therein a plurality of medical images of the subject and identification information of the medical images in a corresponding manner.

2. The medical image processing apparatus according to claim 1, further comprising:
reception circuitry that receives a transmission request for object information; and
transmission circuitry that transmits object information having the transmission request therefor received by the reception circuitry to an external device, wherein
the identification information retrieving circuitry retrieves the identification information from the object information having the transmission request therefor received by the reception circuitry, and
the transmission circuitry transmits a medical image retrieved by the medical image retrieving circuitry with the object information to the external device.

3. The medical image processing apparatus according to claim 1, wherein the object information is configured to be transmittable by DICOM communication.

4. The medical image processing apparatus according to claim 1, further comprising reception circuitry that receives the object information from outside of the medical image processing apparatus, wherein
the identification information retrieving circuitry retrieves the identification information from object information received by the reception circuitry, and
the medical image retrieving circuitry retrieves a medical image corresponding to the identification information retrieved by the identification information retrieving circuitry from the storage circuitry located outside of the medical image processing apparatus.

5. The medical image processing apparatus according to claim 4, wherein the reception circuitry receives the object information from a storage medium, which serves as the outside of the medical image processing apparatus and has information recorded therein in a DICOM media format.

6. A medical image processing method comprising:
retrieving identification information of a medical image serving as a basis of a three-dimensionally displayed image used for diagnosing a subject from object information that includes the identification information and setting information for three-dimensionally displaying the three-dimensionally displayed image; and
retrieving a medical image corresponding to identification information retrieved by the process of retrieving the identification information from storage circuitry that stores therein a plurality of medical images of the subject and identification information of the medical images in a corresponding manner.
